(19) United States
(12) Patent Application Publication
(10) Pub. No.: US 2002/0197274 A1
(43) Pub. Date: Dec. 26, 2002

(54) Lithospermic acid-containing composition

(76) Inventors: Ming-Shi Shiao, Taipei (TW); Yueh-Hsiung Kuo, Taipei (TW); Yun-Lian Lin, Taipei (TW); Yuh-Lien Chen, Taipei (TW); Tsung-Chung Lin, Cheshire, CT (US)

Correspondence Address: Y. ROCKY TSAO, Fish & Richardson P.C., 225 Franklin Street, Boston, MA, 02110-2804, US

(21) Appl. No.: 10/082,548

(22) Filed: Feb. 25, 2002

Related U.S. Application Data
(60) Provisional application No. 60/284,905, filed on Apr. 19, 2001.

(51) Int. Cl. A61K 35/78; A61K 35/72

(52) U.S. Cl. 424/195.16; 424/725; 424/746

(57) ABSTRACT

The present invention relates to a nutraceutical composition that contains lithospermic acid enriched from an extract of T. samentosa, or contains lithospermic acid and salvianolic acid B co-enriched from an extract of S. miltiorrhiza.

### PRIOR APPLICATIONS

This application claims the benefit of prior U.S. provisional application No. 60/284,905, filed Apr. 19, 2001, the contents of which are incorporated herein by reference.

### BACKGROUND

There is a current revival of interest in traditional medicines that are principally derived from Chinese herbs or other types of plants. This interest in Chinese herbs was prompted by Chinese folklore that a number of such herbs have various therapeutic activities and are well tolerated by humans. See Chang et al. (1988) Antiviral Research 9: 163-176, and Kamata et al. (1994); Gen. Pharmac 25: 69-73.

However, traditional Chinese medicines are based largely on anecdotal observations spanning the past several thousands of years. Hence, the effectiveness of herbs used by folk medicine practitioners has, for the most part, not been substantiated by scientific evaluation.

### SUMMARY

This invention relates to polyphenolic acids found in the roots of two herbs Tournefortia samentosa and Salvia miltiorrhiza.

In one aspect, this invention features a method for enriching a polyphenolic acid from T. samentosa or S. miltiorrhiza. A polyphenolic acid includes at least two phenolic rings, e.g., rosmarinic acid (containing two phenolic rings), lithospermic acid and salvianolic acid A (containing three phenolic rings), and salvianolic B (containing four phenolic rings). The method includes the steps of soaking pulverized roots from T. samentosa or S. miltiorrhiza in a sufficient volume of water to dissolve the polyphenolic acid (at an elevated temperature, if necessary); collecting the supernatant; incubating the supernatant with affinity beads which selectively bind to polyphenolic acids; and releasing the polyphenolic acid from the beads with an eluting solvent. The steps of soaking and collecting can be repeated by using one or more volumes of water, if necessary. The polyphenolic acids strongly bind to the affinity beads, while most other compounds either do not bind or only weakly bind to the beads. Optionally, the beads can be rinsed with a washing solvent (e.g., an aqueous solution containing 0-10% ethanol) to remove the compounds that bind to the beads more weakly than the polyphenolic acid to be enriched. The washing step can be repeated. An eluting solvent (e.g., an aqueous solution containing 10-90% ethanol; preferably, 50% ethanol) is used to release the polyphenolic acid from the affinity beads. Water, in a sufficient volume or at an elevated temperature, can also be used to elute the polyphenolic acid from the beads. Lithospermic acid can be enriched from the roots of both T. samentosa and S. miltiorrhiza by the just-described method. When S. miltiorrhiza is used, salvianolic acid B is co-enriched with lithospermic acid.

In another aspect, this invention relates to a nutraceutical composition, which can be a soft drink, milk, a snack bar, juice, a dietary supplement, or a botanical drug. The nutraceutical composition contains 0.05-99% (e.g., 0.05-2% as in a soft drink or 2-99% in a botanical drug) lithospermic acid (or its salt) enriched from an extract of T. samentosa, or contains 0.05-99% (e.g., 0.05-2% as in a soft drink or 2-99% in a botanical drug) lithospermic acid and salvianolic acid B (or its salt) co-enriched from an extract of S. miltiorrhiza. Polyphenolic acids (e.g., lithospermic acid and salvianolic acid B) have been found to be efficacious in treating micro- and macro-vascular disorders, in particular, cardiovascular diseases (e.g., atherosclerosis and restensosis). In other words, an enriched extract of T. samentosa (containing lithospermic acid) or an enriched extract of S. miltiorrhiza (containing both lithospermic acid and salvianolic acid B) is used as an active ingredient in the nutraceutical composition of this invention, which can further include an adequate amount of a cholesterol-lowering agent, such as monacolin K enriched from an extract of red yeast.

This invention further features a pharmaceutical composition that contains lithospermic acid, salvianolic acid B, or a combination of lithospermic acid and salvianolic acid B; and a pharmaceutically acceptable carrier. Optionally, the pharmaceutical composition also includes a cholesterol-lowering agent. Each of lithospermic acid and salvianolic acid B can be either synthesized from organic chemicals or purified from a natural source, and each of them can be in the form of a pharmaceutically acceptable salt. For example, a salt can be formed between a negatively charged group such as carboxylate in lithospermic acid or salvianolic acid B and a positively charged counterion such as an alkali metal ion (e.g., a sodium ion or a potassium ion); an alkaline earth metal ion (e.g., a magnesium ion or a calcium ion); an ammonium ion (NH₄⁺); or an organic ammonium group such as tetramethylammonium ion or diisopropylethyl-ammonium ion. The present invention also encompasses the use of lithospermic acid, salvianolic acid B, or a combination of both for the manufacture of a medicament for treating atherosclerosis or restenosis.

Also within the scope of this invention is a method of treating atherosclerosis or restenosis. The method includes administering to a subject in need of such treatment an effective amount of lithospermic acid, salvianolic acid B, or a combination of both. The method may further include concurrently administering to the subject an effective amount of a cholesterol-lowering agent.

Other advantages or features of this invention will be apparent from the following detailed description thereof.

### DETAILED DESCRIPTION

A large number of compounds, such as alkaloids, flavones, phenolics, and cinnamates, have been found in the roots of T. samentosa and S. miltiorrhiza. For example, over 50 compounds, not including fatty acids, amino acids, and nucleosides, have been isolated from S. miltiorrhiza, including 1,2,15,16-tetrahydro-tanshiquinone, tanshinaldehyde, Ro-090680, dihydro-isotanshone I, danshexinkun B, miltirone, nortanshinone, hydroxytanshinone II-A, tanshinone I, dihydrotanshinone I, tanshinone II-A, cryptotanshinone, methylenetanshiquinone, methyltanshinonate, spiroketallactone, neocrypto-tanshinone, isotanshinone IIB, danshexinkun A, danshenol A, danshenol B, protocatechualdehyde, caffeic acid, methyl rosmarinate, rosmarinic acid, salvianolic acid A, salvianolic acid B, salvianolic acid C, and lithospermic acid. See Men'shikov et al. (1952) J. Gen. Chem. 22: 1465-1467; Delorme et al. (1977) Plant Med. Phytother. 11:5-11; Ogihara et al. (1997) Bull. Coll. Sci. 64: 53-59; Lin et al. (1999) J. Nat. Prod. 62: 1500-1503; Crowley & Culvenor (1955) J. Aust. J. Chem. 8: 464-465; Li et al. (1984) Planta. Medica. 50: 227-228; Lee et al. (1987) J. Nat. Prod. 50: 157-160; and Ai et al. (1988) J. Nat. Prod. 51: 145-149.

This invention is based on the discovery that lithospermic acid (present in both T. samentosa and S. miltiorrhiza), as well as salvianolic acid B present in S. miltiorrhiza), possesses anti-atherosclerosis and anti-restensosis activities. Thus, a nutraceutical composition of this invention contains lithospermic acid as a component of an enriched extract of T. samentosa, or contains lithospermic acid and salvianolic acid B as components of an enriched extract of S. miltiorrhiza. Typically, lithospermic acid (as well as other polyphenolic acids), can be enriched from roots of T. samentosa or S. miltiorrhiza, and salvianolic B (as well as other polyphenolic acids) can be enriched from roots of S. miltiorrhiza by the following procedure. T. samentosa or S. miltiorrhiza roots are pulverized and soaked in water. The resultant dispersion is incubated at 60-100° C. for 1-2 hr. The dispersion is centrifuged or filtered and its supernatant collected. The volume of water, the incubation temperature, and the incubation time can be adjusted and optimized in order to achieve maximum extraction and yield of the polyphenolic acid to be enriched. For example, heating (e.g., 70° C.) for a short time (e.g., 2 hr) may facilitate extraction of the polyphenolic acids from the roots without causing decomposition thereof. The soaking, incubating, and collecting steps can be repeated by using one or more volumes of water. The supernatants can be combined and mixed with affinity beads, which selectively bind to polyphenolic acids. An example of such beads is HP-20 styrene beads (DIAION beads, Mitsubishi, Japan). The beads can be rinsed with a washing solvent (e.g., an aqueous solution containing 0-10% ethanol) to remove compounds that bind more weakly to the beads than polyphenolic acids. The washing step can be repeated. The polyphenolic acid is then released with an eluting solvent (e.g., an aqueous solution containing 10-90% ethanol; preferably, 50% ethanol). The eluent, which contains the polyphenolic acid to be enriched, is collected and dried.

When lithospermic acid is enriched from S. miltiorrhiza by the above described method, salvianolic acid B is co-enriched. An enriched extract of T. samentosa containing lithospermic acid or an enriched extract of S. miltiorrhiza containing lithospermic acid and salvianolic acid B thus obtained is used to prepare a nutraceutical composition of this invention for treating (including preventing) atherosclerosis or restenosis. The nutraceutical composition can be a dietary supplement (e.g., a capsule, a mini-bag, or a tablet), a food product (e.g., a soft drink, milk, juice, a herbal tea-bag, or confectionary), or a botanical drug. The dietary supplement may also include other nutrients, such as protein, carbohydrate, vitamins, minerals, or amino acids. The botanical drug can be in a form suitable for oral use, such as a tablet, a hard or soft capsule, an aqueous or oil suspension, or a syrup; or in a form suitable for parenteral use, such as an aqueous propylene glycol solution, or a buffered aqueous solution. The amount of the active ingredient in the nutraceutical composition depends to a large extent on a subject's specific need. The amount will also vary, as recognized by those skilled in the art, dependent on administration route, and possible co-usage of other anti-atherosclerosis or anti-restensosis agents.

Also within the scope of this invention is a pharmaceutical composition that contains an effective amount of lithospermic acid or salvianolic acid B (or both) for treating atherosclerosis or restenosis, and a pharmacentically acceptable carrier. The pharmaceutical composition may further include an effective amount of a cholesterol-lowering agent. Lithospermic acid and salvianolic acid B can be prepared by synthetic methods or purified from a natural source. For example, lithospermic acid in an enriched extract of T. samentosa can be purified by high pressure liquid chromatography. The pharmaceutically acceptable carrier includes a solvent, a dispersion medium, a coating, an antibacterial and antifungal agent, an isotonic and absorption delaying agent. An “effective amount” is the amount required to confer therapeutic effect. The interrelationship of dosages for animals and humans (based on milligrams per meter squared of body surface) is described by Freireich et al. (1966) Cancer Chemother. Rep. 50: 219. Body surface area may be approximately determined from height and weight of the patient. See, e.g., Scientific Tables, Geigy Pharmaceuticals, Ardley, N.Y., 1970, 537. Effective doses will also vary, as recognized by those skilled in the art, dependent on route of administration, excipient usage, and the like.

Lithospermic acid or salvianolic acid B can be formulated into dosage forms for different administration routes utilizing conventional methods. For example, it can be formulated in a capsule, a gel seal, or a tablet for oral administration. Capsules may contain any standard pharmaceutically acceptable materials such as gelatin or cellulose. Tablets may be formulated in accordance with conventional procedures by compressing mixtures of lithospermic acid or salvianolic acid B with a solid carrier and a lubricant. Examples of solid carriers include starch and sugar bentonite. Lithospermic acid or salvianolic acid B can also be administered in a form of a hard shell tablet or a capsule containing a binder, e.g., lactose or mannitol, a conventional filler, and a tableting agent. The pharmaceutical composition may be administered via the parenteral route. Examples of parenteral dosage forms include aqueous solutions, isotonic saline or 5% glucose of the active agent, or other well-known pharmaceutically acceptable excipient. Cyclodextrins, or other solubilizing agents well-known to those familiar with the art, can be utilized as pharmaceutical excipients for delivery of the therapeutic compound.

An in vitro assay can be used to evaluate the efficacy of a composition of this invention in inhibiting low density lipoprotein (LDL) oxidation. Such inhibition interrupts the progression of atherosclerosis, as well as halts the process of restenosis after angioplasty. See Lusis et al. (1993) Biochem Pharmacol. 46: 2119-2126; and Faxon et al. (1997) Prog. Cardiovasc. Dis. 40: 129-140). For example, the composition to be evaluated can be dissolved in a phosphate buffer, and added to a pre-incubated LDL-containing solution. Subsequently, LDL oxidation reaction is initiated with addition of a CuSO₄ solution. After incubation, the reaction is quenched with an EDTA solution. Then, the supernatant is collected and its UV absorption measured to determine the amount of oxidized-LDL, a conjugated diene. The anti-LDL oxidation activity is evaluated based on the amount of the conjugated diene.

Another in vitro assay can be used to evaluate the efficacy of a composition of this invention in inhibiting the expression of intercellular cell adhesion molecule-1 (ICAM-1) or vascular cell adhesion molecule-1 (VCAM-1). Such inhibition prevents leukocyte recruitment to the sites of atherosclerosis. Van der Wal et al. (1992) Am. J. Pathol. 141:1427-1433.

A composition of this invention can be further tested in animal studies. For example, apoE-deficient mice (obtained from Jackson Laboratory, Bar Harbor, Me., USA) are used and divided into three different groups. Mice in the control group are fed with a diet, for example, containing 0.15% cholesterol (w/w) (a control diet). A test group is fed with the control diet further containing a composition of this invention. For comparison, a compound such as probucol or N,N′-diphenyl-phenylenediamine (DPPD), being demonstrated to reduce atherosclerosis in cholesterol-fed animals, is included in another diet fed to another test group of mice. After a period of time, the atherosclerotic lesion area in mice of each group is determined in the arch, thoracic, and abdominal. The anti-atherosclerosis effect of the composition is evaluated based on the degree of the reduction of the lesion, compared with that of probucol or DPPD. Different dosages and administration routes can be tested. Based on the results, an appropriate dosage range and administration route can be determined.

In vivo tests can also be performed on rabbits. For example, New Zealand White rabbits (body weight 2.5-2.8 kg) are randomly divided into three groups. One group of rabbits is fed with a diet, for example, containing 2% cholesterol (a control diet). Another group is fed with the control diet containing a compound such as probucol and the third group is fed with the control diet further containing a composition of this invention. After being fed a period of time, all rabbits undergo an endothelial balloon injury, and the diet fed to each group is maintained for another period of time. The anti-restenosis effect of the composition is evaluated by comparison with that of probucol. Different dosages and administration routes can be tested. Based on the results, an appropriate dosage range and administration route can be determined.

Without further elaboration, it is believed that one skilled in the art can, based on the description herein, utilize the present invention to its fullest extent. All publications cited herein are hereby incorporated by reference in their entirety.

### OTHER EMBODIMENTS

All of the features disclosed in this specification may be combined in any combination. Each feature disclosed in this specification may be replace by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, compounds structurally analogous to lithospermic acid or salvianolic acid B also can be made, screened for their anti-atherosclerosis or anti-restenosis activities, and used to practice this invention. Thus, other embodiments are also within the claims.

What is claimed is:

1. A nutraceutical composition comprising 0.05-99% by weight lithospermic acid, wherein said lithospermic acid is enriched from an extract of Tournefortia sarmentosa.

2. The nutraceutical composition of claim 1, wherein the composition comprising 0.05-2% by weight the enriched lithospermic acid.

3. The nutraceutical composition of claim 1, wherein the composition comprising 2-99% by weight the enriched lithospermic acid.

4. The nutraceutical composition of claim 1, further comprising a cholesterol-lowering agent.

5. The nutraceutical composition of claim 4, wherein the cholesterol-lowering agent is a monacolin K-containing extract of red yeast.

6. A nutraceutical composition comprising 0.05-99% by weight lithospermic acid and salvianolic acid B co-enriched from an extract of Salvia miltiorrhiza.

7. The nutraceutical composition of claim 6, wherein the composition comprises 0.05-2% by weight the co-enriched lithospermic acid and salvianolic acid B.

8. The nutraceutical composition of claim 6, wherein the composition comprises 2-99% by weight the co-enriched lithospermic acid and salvianolic acid B.

9. The nutraceutical composition of claim 6, further comprising a cholesterol-lowering agent.

10. The nutraceutical composition of claim 9, wherein the cholesterol-lowering agent is a monacolin K-containing extract of red yeast.

11. A method of enriching a polyphenolic acid from Tournefortia sarmentosa or Salvia miltiorrhiza, the method comprising:
soaking pulverized roots from Tournefortia sarmentosa or Salvia miltiorrhiza in water to dissolve the polyphenolic acid;
collecting the supernatant;
incubating the supernatant with affinity beads which selectively bind to the polyphenolic acid; and
releasing the polyphenolic acid from the beads with an eluting solvent.

12. The method of claim 11, wherein the polyphenolic acid contains two phenolic rings.

13. The method of claim 11, wherein the polyphenolic acid contains three phenolic rings.

14. The method of claim 11, wherein the polyphenolic acid contains four phenolic rings.

15. The method of claim 11, wherein the polyphenolic acid is lithospermic acid.

16. The method of claim 15, wherein lithospermic acid is enriched from Tournefortia sarmentosa or Salvia miltiorrhiza.

17. The method of claim 11, wherein the polyphenolic acid is salvianolic acid B.

18. The method of claim 17, wherein salvianolic acid B is enriched from Salvia miltiorrhiza.
